(19) United States
(12) Patent Application Publication
(10) Pub. No.: US 2005/0002958 A1
(43) Pub. Date: Jan. 6, 2005

(54) Vaccines

(75) Inventors: Joseph Cohen, Rixensart (BE); Nathalie Garcon, Rixensart (BE); Gerald Voss, Rixensart (BE)

Correspondence Address: GLAXOSMITHKLINE;Corporate Intellectual Property - UW2220, P.O. Box 1539, King of Prussia, PA, 19406-0939, US

(73) Assignee: SmithKline Beecham Biologicals SA

(21) Appl. No.: 10/789,758

(22) Filed: Feb. 27, 2004

Related U.S. Application Data
(63) Continuation of application No. 10/018,704, filed on Mar. 7, 2002, now abandoned.

(30) Foreign Application Priority Data
Jun. 29, 1999 (GB) 9915204.3

(51) Int. Cl. A61K 48/00; A61K 39/002

(52) U.S. Cl. 424/191.1; 514/44

(57) ABSTRACT

A vaccine formulation for the prevention or amelioration of plasmodium infection in humans is provided. The vaccine comprises a malaria antigen, especially a protein which comprises a portion of the CS protein of P. falciparum fused in frame via a linear linker to the N-terminal of HBsAg, and an immunostimulatory CpG oligonucleotide. Methods for making the vaccine formulation of the invention are described. Patients may also be treated by pre-administration of the CpG oligonucleotide prior to administration of the malaria antigen.

The present invention relates to a novel vaccine formulations and their use in medicine, particularly in the prevention of malaria infections. In particular the present invention is concerned with a CpG oligonucleotide and a malarial antigen.

Malaria, is one of the world's major health problems with 2 to 4 million people dying from the disease each year. One of the most acute forms of the disease is caused by the protozoan parasite, Plasmodium falciparum which is responsible for most of the mortality attributable to Malaria.

The life cycle of P. falciparum is complex, requiring two hosts, man and mosquito for completion. The infection of man is initiated by the inoculation of sporozoites in the saliva of an infected mosquito. The sporozoites migrate to the liver and there infect hepatocytes where they differentiate, via the exoerythrocytic intracellular stage, into the merozoite stage which infects red blood cells (RBC) to initiate cyclical replication in the asexual blood stage. The cycle is completed by the differentiation of a number of merozoites in the RBC into sexual stage gametocytes which are ingested by the mosquito, where they develop through a series of stages in the midgut to produce sporozoites which migrate to the salivary gland.

The sporozoite stage of P. falciparum has been identified as a potential target of a malaria vaccine. The major surface protein of the sporozoite is known as circumsporozoite protein (CS Protein). This protein from strain 7G8 has been cloned, expressed and sequenced (Dame et al Science 225 (1984) p593). The protein from strain 7G8 is characterised by having a central immunodominant repeat region comprising a tetrapeptide Asn-Ala-Asn-Pro repeated 37 times but interspersed with four minor repeats Asn-Val-Asp-Pro. In other strains the number of major and minor repeats vary as well as their relative position. This central portion is flanked by an N and C terminal portion composed of non-repetitive amino acid sequences designated as the repeatless portion of the CS protein.

It has been shown that irradiated sporozoites can provide significant protection against experimental human malaria (Am. J. Trop. Med. Hyg. 24: 297-402, 1975). However, production difficulties makes the use of irradiated sporozoite impractical from the point of view of producing a vaccine.

Several groups have proposed subunit vaccines based on the circumsporozoite protein. Two of these vaccines have undergone clinical testing; one is a synthetic peptide, the other is a recombinant protein (Ballou et al Lancet: i 1277 (1987) and Herrington et al Nature 328:257 (1987).

These vaccines were successful in stimulating an anti-sporozoite response. Nonetheless, the magnitude of the response was disappointing, with some vaccinees not making a response at all. Furthermore, the absence of “boosting” of antibody levels on subsequent injections and results of in vitro lymphocyte proliferation assays suggested that T-cells of most of these volunteers did not recognise the immuno-dominant repeat. Nonetheless, one vaccinee in each study did not develop parasitemia.

The present invention provides a new, improved malaria vaccines which not only produces a humoral response, but also a cellular immune response. Preferably the antigen induces the production of neutralising antibodies against the immunodominant repeat. Most preferably, the antigen should also elicit effector T cell mediated immune responses of the CD4⁺ and CD8⁺ cytotoxic T lymphocyte (CTL) type and of the delayed type hypersensitivity type and also, preferably be able to induce T helper (TH) memory cells.

International patent application No. WO 93/10152 (SmithKline Beecham Biologicals s.a) provides a hybrid protein comprising substantially all the C-terminal portion of the CS protein, four or more tandom repeats of the immunodominant region, and the Surface antigen from Hepatitis B virus (HBsAg). Preferably the hybrid protein comprises a sequence which contains at least 160 amino acids which is substantially homologous to the C-terminal portion of the CS protein. The CS protein may be aevoid of the last 12 amino-acids from the C terminal.

In particular there is provided a protein which comprises a portion of the CS protein of P. falciparum substantially as corresponding to amino acids 210-398 of P. falciparum 7G8 fused in frame via a linear linker to the N-terminal of HBsAg. The linker may comprise a portion of preS2 from HBsAg.

A particularly preferred embodiment is the hybrid protein designated RTS (or RTS,S). This hybrid consists of:
A methionine-residue, encoded by nucleotides 1059 to 1061, derived from the Saccharomyces cerevisiae TDH3 gene sequence. (Musti A.M. et al Gene 1983 25 133-143).
Three amino acids, Met Ala Pro, derived from a nucleotide sequence (1062 to 1070) created by the cloning procedure used to construct the hybrid gene.
A stretch of 189 amino acids, encoded by nucleotides 1071 to 1637 representing amino acids 210 to 398 of the circumsporozoite protein (CSP) of Plasmodium falciparum strain 7G8 (Dame et al supra).
An amino acid (Arg) encoded by nucleotides 1638 to 1640, created by the cloning procedure used to construct the hybrid gene.
Four amino acids, Pro Val Thr Asn, encoded by nucleotides 1641 to 1652, and representing the four carboxy terminal residues of the hepatitis B virus (adw serotype) preS2 protein (9).
A stretch of 226 amino acids, encoded by nucleotides 1653 to 2330, and specifying the S protein of hepatitis B virus (adw serotype).

In an alternative embodiment there is provided a hybrid protein designated RTS* (or RTS*,S), which was generated using the CSP gene sequence from P. falciparum NF54 (Mol. Biochem Parisitol. 35: 185-190, 1989) and comprises substantially all of the region 207 to 395 of the CS protein from P falciparum NF54.

In particular RTS* comprises:
A Methionine, encoded by nucleotides 1059 to 1061, derived from the TDH3 gene sequence (see Musti et al, loc cit).
Three amino acids, Met Ala Pro, derived from a nucleotide sequence (1062 to 1070) created by the cloning procedure used to construct the hybrid gene.
A stretch of 189 amino acids, encoded by nucleotides 1071 to 1637 representing amino acids 207 to 395 of the circumsporozoite protein (CSP) of Plasmodium falciparum strain NF54 (Mol.Biochem.Parasitol, 35:185-190, 1989).
An amino acid (Gly) encoded by nucleotides 1638 to 1640, created by the cloning procedure used to construct the hybrid gene.
Four amino acids, Pro Val Thr Asn, encoded by nucleotides 1641 to 1652, and representing the four carboxy terminal residues of the hepatitis B virus (adw serotype) preS2 protein (Nature 280:815-819, 1979).
A stretch of 226 amino acids, encoded by nucleotides 1653 to 2330, and specifying the S protein of hepatitis B virus (adw serotype) (Nature 280:815-819, 1979)

International patent application no. WO 90/01496 describe an antigen known as Trap (or TRAP) from P. falciparum. An apparent homologue of Trap is described in WO92/11868 and relates to an antigen called SSP2 from P. yeolii.

International patent application WO 98/ 05355 describes, inter alia, a malaria vaccine based on a combination of Trap and RTS,S.

Immunomodulatory oligonucleotides contain unmethylated CpG dinucleotides (“CpG”) and are known (WO 96/02555, EP 468520). CpG is an abbreviation for cytosine-guanosine dinucleotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546 1995. Detailed analysis has shown that the CG motif has to be in a certain sequence context, and that such sequences are common in bacterial DNA but are rare in vertebrate DNA.

It is currently believed that this evolutionary difference allows the vertebrate immune system to detect the presence of bacterial DNA (as occurring during an infection) leading consequently to the stimulation of the immune system. The immunostimulatory sequence as defined by Krieg is:

Purine Purine CG pyrimidine pyrimidine and where the CG motif is not methylated. In certain combinations of the six nucleotides a palindromic sequence is present. Several of these motifs, either as repeats of one motif or a combination of different motifs, can be present in the same oligonucleotide. The presence of one or more of these immunostimulatory sequence containing oligonucleotides can activate various immune subsets, including natural killer cells (which produce interferon γ and have cytolytic activity) and macrophages (Wooldrige et al Vol 89 (no. 8), 1977). Although other unmethylated CpG containing sequences not having this consensus sequence have now been shown to be immunomodulatory.

The present invention provides an improved vaccine formulation comprising a CpG oligonucleotide and a malaria antigen. In particular, RTS,S or RTS,S* or Trap or immunologically equivalent derivatives thereof.

Vaccine preparation is generally described in Vaccine Design—The subunit and adjuvant approach Ed. Powell and Newman) Pharmaceutical Biotechnology Vol. 6 Plenum Press 1995. Encapsulation within liposomes is described by Fullerton, U.S. Pat. No. 4,235,877.

The preferred oligonucleotides preferably contain two or more CpG motifs separated by six or more nucleotides. The oligonucleotides of the present invention are typically deoxynucleotides. In a preferred embodiment the internucleotide in the oligonucleotide is phosphorodithioate, or more preferably a phosphorodithioate bond, although phosphodiester and other internucleotide bonds are within the scope of the invention including oligonucleotides with mixed intemucleotide linkages. The sequences preferably contain all phosphorodithioate modified internucleotide linkages. Preferred oligonucleotides have the following sequences:

| Oligo (internal | | | |
|---|---|---|---|
| designation*) | 5′-SEQUENCE-3′ | CpG | Thio |
| WD1001 | TCC ATG ACG TTC CTG ACG TT | + | + |
| WD1002 | TCT CCC AGC GTG CGC CAT | + | + |
| WD1003 | ACC GAT AAC GTT GCC GGT GAC G | + | − |
| WD1004 | G*G*G GTC AAC GTT GAG* G*G*G*G*G | + | Mix |
| WD1005 | TCC ATG AGC TTC CTG AGC TT | − | + |
| WD1006 | TCC ATG ACG TTC CTG ACG TT | + | − |
| WD1007 | ACC GAT GAC GTC GCC GGT GAC GGC ACC ACG | + | + |
| | TCG TCG TTT TGT CGT TTT GTC GTT | + | + |

*alternatively referred to as WD001-WD007

In the above table a + in the Thio column indicates the presence of a thioate modification. ‘Mix’ indicates a mixture of thioate modification and sequence without thioate modification (the asterisks indicate the linkages with a thioate modification). A − in the Thio column indicates absence of a thioate modification. A + in the CpG column indicates a the presence of a CpG motif and a − in the CpG column indicates absence of a CpG motif. For example WD1005 contains a GpC rather than a CpG motif, thus it is marked with a − in the CpG column of the table. WD1007 contains a palindromic motif (GACGTC) as well as other non-palindromic CpG sequences. This is also within the scope of a CpG oligonucleotide as the term is used in the present application.

The oligonucleotides utilised in the present invention may be synthesized by any method known in the art (eg EP 0 468 520). Conveniently, such oligonucleotides may be synthesized utilising an automated synthesizer. Methods for producing phosphorothioate oligonucleotides or phosphorodithioate are described in U.S. Pat. No. 5,666,153, U.S. Pat. No. 5,278,302 and WO95/26204.

The amount of protein in each vaccine does is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 2-100 μg, most preferably 5-50 μg. An optimal amount for a particular vaccine can be ascertained by standard studies involving observation of appropriate immune responses in subjects. Following an initial vaccination, subjects may receive one or several booster immunisations adequately spaced.

It is also possible to pre-administer the CpG oligonucleotide as a ‘priming formulation’ shortly prior to vaccination with the malaria antigen, for example 1 day before.

Accordingly, according to another aspect of the invention, there is provided a method for the prevention or amelioration of plasmodium infection in a patient, comprising administering an effective amount of either a malaria antigen and a CpG oligonucleotide (as hereinabove defined) or an effective amount of the CpG oligonucleotide followed after a suitable time by an effective amount of a malaria antigen.

There is also provided a kit comprising effective amounts of a CpG oligonucleotide-containing formulation for use as a priming formulation for pre-administration to human patients and a malaria antigen for injection at some suitable time later, as described hereinabove.

Preferred CpG oligonucleotides are those indicated in the table hereinabove.

Suitably the CpG will be present in the range 10 μg per dose to 1000 μg, preferably 10-100 μg, especially 25-75 μg, for example 50 μg per dose.

Suitably the vaccine used in the present invention may comprise a carrier such as an aluminium salt, eg aluminium hydroxide [Al(OH)3], aluminium phosphate or aluminium phosphate sulfate (alum), or a non-toxic oil in water emulsion or a mixture thereof.

If an aluminium salt (preferably aluminium hydroxide) is used as a carrier it is generally present in the range of 50 to 100 μg, preferably 100 to 500 μg per dose.

Non-toxic oil in water emulsions preferably contain a non-toxic oil, eg squalene and an emulsifier such as (polysorbitan monoleate) Tween 80, in an aqueous carrier such as phosphate buffered saline.

If desired the vaccine used in the present invention may comprise an additional adjuvant, preferably a saponin adjuvant such as QS21 as described for example in WO 9517210, optionally in the presence of a sterol, such as cholesterol as described for example in PCT/EP96/01464. The vaccine of the invention may also comprise monophosphoryl lipid A and derivatives thereof known in the art. A preferred derivative is 3 de-O-acylated monophosphoryl lipid A, described in British Patent No. 2220211.

Accordingly vaccine formulations of the present invention may additionally comprise other pharmaceutical excipients or immunostimulants. In a preferred embodiment the vaccine formulation additionally comprises an aluminium salt, preferably aluminium hydroxide.

The present invention will now be described with reference to the following examples:

### IMMUNOGENICITY STUDIES USING RTS,S FORMULATED WITH CPG OR CPG/ALUM

Evaluation of CpG and CpG/alun in Mice

Experiment Outline

An immunogenicity study was conducted to evaluate the ability of CpG to serve as an adjuvant for cytotoxic T lymphocyte (CTL) induction. Groups of mice were immunized with RTS,S formulated with CpG oligonucleotide alone or in combination with aluminum hydroxide. After two immunizations spleen cells were examined for the presence of HbsAg-specific effector cells.

TABLE 1
Groups of mice

| Group | antigen | adjuvant |
|---|---|---|
| 1 | RTS, S | CpG/alum |
| 2 | RTS, S | CpG |

Formulation

Component Batches Used:

| | | | CON- | |
|---|---|---|---|---|
| | | BATCH | CENTRATION | |
| COMPONENT | BRAND | NUMBER | (MG/ML) | BUFFER |
| RTSS | | 24R51 | 0.664 | P/N 6.8 |
| Al(OH)₃ | Superfos | 97A0027 | 10.380 | H₂O |
| CpG | Eurogen | 324255R1 | 5 | H₂O |
| (WD1001) | etech | | | |

Formulation Process:

Formulations were prepared 3 days before each injection. All incubations were carried out at room temperature with agitation.

CpG/alum Group 1 (500,μl/dose)

RTS, S (8.7 μg) and gp120 (8.7 μg) were adsorbed on 100 μg of Al(OH)₃ or AlPO₄ for 1 hour The formulation was buffered with a 10-fold concentrated PO₄/NaCl pH 6.8 solution before addition of 100 μg of CpG (WD1001). After 15 min, 50 μg/ml of thiomersal was added as preservative.

CpG Group 2 (500 μl/dose)

RTS,S (8.7 μg) and Gp120(8.7 μg) were diluted in PBS pH 6.8 before addition of 100 μg of CpG (WD1001). After 5 min, 50 μg/ml of thiomersal was added as preservative.

Immunological Methods

Nine Balb/C mice per group received into the hind footpads 100 μl vaccine twice at a two-week-interval. Two weeks later spleen cells were harvested and used to determine the induction of HBsAg-specific CTL.

For CTL analysis cells were cultured for 7 days in 6-well plates in the presence of 10 μg per ml of synthetic peptide pCMI003 corresponding to an HBsAg CTL epitope (Schirmbeck et al., 1995). At the end of the culture period cells were assessed in duplicate for HBsAg-specific cytolytic activity in standard [⁵¹Cr]-release assays using control and S-transfected P815 cells. Minimum and maximum release were determined with target cells without effector cells and by the addition of 3% (v/v) Triton X-100, respectively. Results are expressed as % [51Cr]-release (cpm of exp. culture—cpm of spont. release/cpm of max. release—cpm of spont. release).

Results

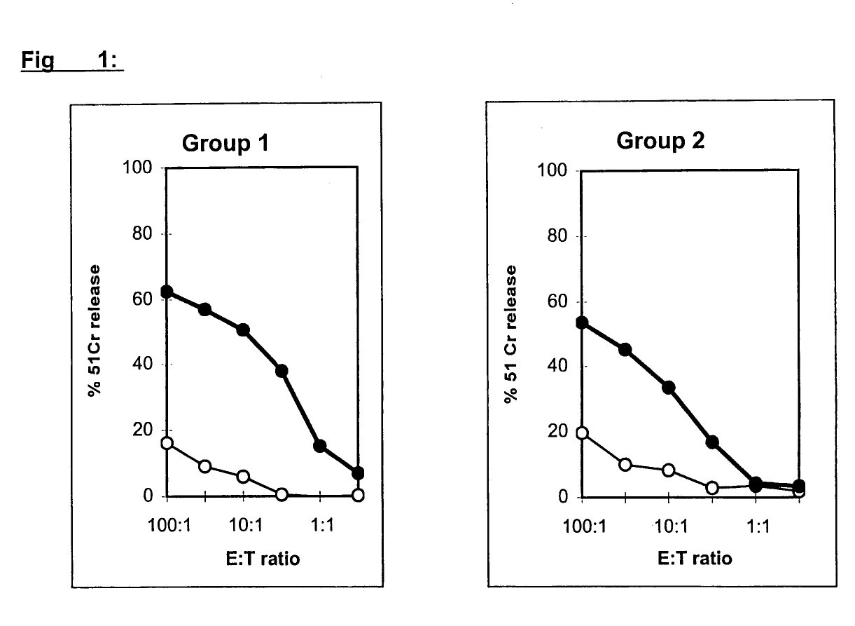

Spleen cells from both groups of mice exhibited HBsAg-specific effector cell function in ⁵¹Cr release assays (FIG. 1). Untransfected P815 target cells were lysed to a much lesser degree than similar target cells expressing the HBV s gene. Lysis of the target cells diminished with decreasing effector to target cell ratios.

Conclusions

Immunization with RTS,S in combination with CpG or CpG/alum induces HBsAg-specific CTL in mice.

### EVALUATION OF CPG AND CPG/ALUM IN RHESUS MONKEYS

Experiment Outline

An immunogenicity study was conducted to evaluate the adjuvant effect of CpG in non-human primates. Groups of five monkeys were immunized twice with RTS,S in combination with CpG or CpG/alum. After the second immunization the immune response of the animals was assessed. Antibodies to HBsAg and lymphoproliferative as well as cytokine responses were evaluated.

TABLE 2
Groups of monkeys

| Group | antigen | adjuvant |
|---|---|---|
| 1 | RTS, S | CpG/alum |
| 2 | RTS, S | CpG |

Formulation

Component Batches Used:

| | | | CON- | |
|---|---|---|---|---|
| | | BATCH | CENTRATION | |
| COMPONENT | BRAND | NUMBER | (MG/ML) | Buffer |
| RTS,S | | ERTS1X058 | 1.372 | P/N 6.8 |
| Al(OH)₃ | Superfos | 96A0089 | 10.380 | H₂O |
| CpG | | WD1001 | 5 | H₂O |

Formulation Process:

Formulations were prepared one day before each injection. All incubations were carried out at room temperature with agitation.

CpG/alum group 1 (500 μl /dose)

RTS,S (50 μg) was adsorbed on 500 μg of Al(OH)₃ for 1 hour The formulation was buffered with a 10-fold concentrated PO₄/NaCl pH 6.8 solution before addition of 500 μg of CpG (WD1001). After 15 min, 50 μg/ml of thiomersal was added as preservative.
H₂O+Al(OH)₃+RTS ,S−1H−10×PN−15m−CpG−15m−Thio
GCpG Group 2 (500 μl/dose)

RTS,S (50 μg) was diluted in PO₄/NaCl buffer pH 6.8 before addition of 500 μg of CpG WD1001. After 15 min, 50 μg/ml of thiomersal was added as preservative.
H₂O+RTSS+10×PN−15m−CpG−15m−Thio
Immunological Methods

Five rhesus monkeys (Macaca mulatta) per group were immunized twice intramuscularly with 500 μl of vaccine at a four-week-interval. Sera and peripheral blood mononuclear cells (PBMC) were taken at several occasions.

HBsAg-specific antibodies in monkey sera were determined in a radio immuno assay (RIA, Abbott) according to the manufacturer's instructions.

Lymphoproliferation was assessed by using density gradient-purified PBMC from immunized rhesus monkeys. Cells were seeded in quadruplicates at 1×10⁵ in 100 μl RPMI/5% FCS per well in round bottom 96 well plates. Then another 100 μl of medium alone or containing soluble RTS,S (10 μg/ml) were added and parallel cultures were incubated for 48 hrs. Thereafter, 100 μl culture supernatant were replaced by fresh medium containing 1 μCi [³H]-thymidine. After 16 hrs cells were harvested onto filter plates and incorporated radioactivity was determined in a β-counter. Results are expressed in cpm and in stimulation indices (SI,=cpm antigen-containing cultures/cpm medium alone cultures), SI greater than 3 are considered as a positive response.

Flat bottom 96 well plates were prepared by coating an IFN-γ-specific capture antibody in 50 μl PBS for 4 hrs at 37° C. The plates were washed three times and PBMC were seeded similar to lymphoproliferation assays. After 48 hrs of culture the plates were washed thrice with PBS/0.05% Tween 20 and 50 μl of biotinylated secondary IFN-γ-specific antibody diluted in PBS/Tween/1% FCS were added for 2 hrs. The plates were washed again and a gold-conjugated α-biotin antibody was incubated for 1 hr. After additional washings the ELIspots were visualized by using a silver enhancing kit (50 μl per well). The reaction was stopped after approx. 30 min by adding deionized water. Cytokine-secreting cells were enumerated by microscopic examination.

Results

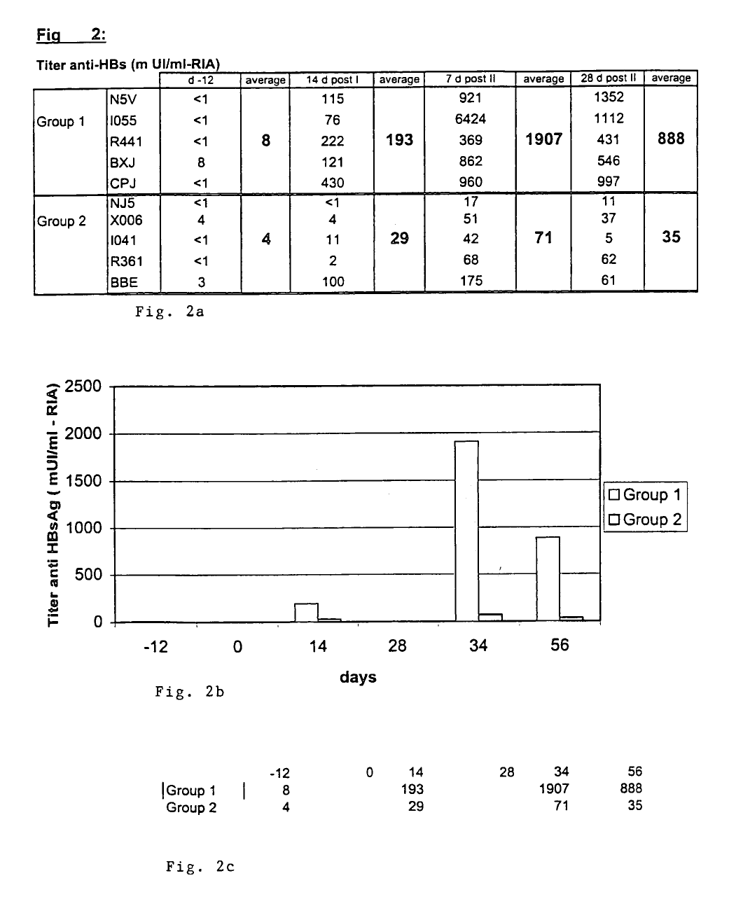

Analysis of HBsAg-specific antibodies in sera of the monkeys revealed that all animals in the two groups had developed specific immune responses (FIG. 2). Some responses were detectable already after one immunization. Interestingly, these responses were boosted by the second immunization only in group 1, while titers in group 2 remained more or less constant.

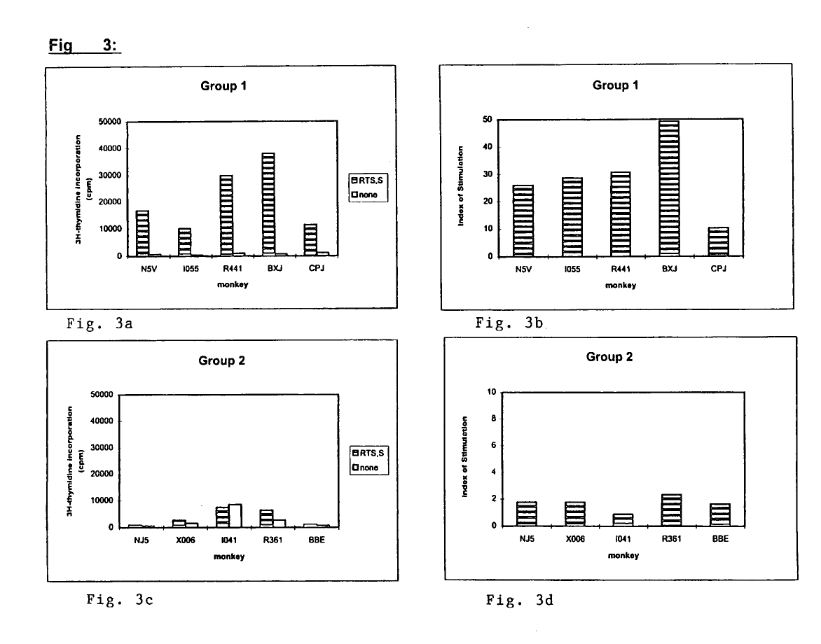

Induction of specific lymphoproliferation by immunization with RTS,S in combination with CpG or CpG/alum was evaluated before immunization and 6 days post secondary immunization. All 10 animals did not exhibit any specific lymphoproliferation (SI>3) at the study start (data not shown). In contrast, all animals in group 1 possessed strong lymphoproliferative responses 6 days post boost immunization (FIG. 3). All animals from group 2 did, however, remain negative in this analysis.

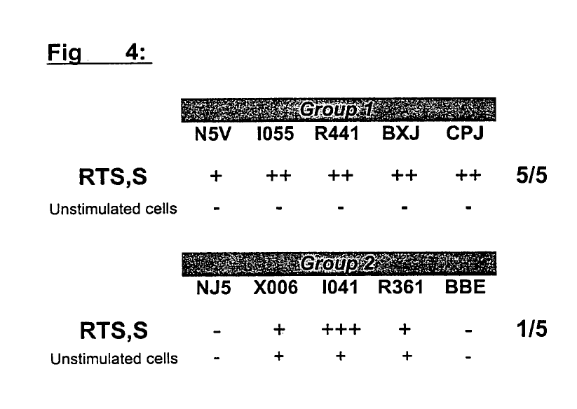

The presence of RTS,S-specific IFN-γ-secreting cells was investigated in all monkeys before immunization and 6 days after the second dose. IFN-γ-secreting cells could not be evaluated from pre-immunization samples due to technical difficulties. However, such cells were detectable after secondary immunization (FIG. 4). All animals in group 1 exhibited a positive response, while only one animal in group 2 was positive.

Conclusions

Immunization with RTS,S in combination with CpG induces immune responses in non-human primates. After two immunizations CpG alone induces low level HB sAg-specific antibodies, while CpG combined with alum induces high titer antibodies as well as vigorous lymphoproliferative and IFN-γ responses.

Figure Legends

FIG. 1: CTL activity of spleen cells from immunized mice. Effector cell activity was assessed by examining ⁵¹Cr release of P815 cells (open circles) or s-transfected P815 cells (closed circles).

FIG. 2: HBsAg-specific antibody responses in immunized rhesus monkeys. Specific antibodies were evaluated using a commercially available RIA. Individual values from multiple time points for each animal are shown in the table, and group averages are shown in the table and as a graphic.

FIG. 3: RTS,S-specific lymphoproliferation in immunized rhesus monkeys 6 days post second immunization. PBMC were stimulated with RTS,S antigen and lymphoproliferative responses were measured by ³H-thymidine incorporation. Results are expressed in cpm and as SI.

FIG. 4: RTS,S-specific IFN-γ-secreting cells from immunized rhesus monkeys. IFN-γ-secreting cells were visualized by the ELIspot method. Cytokine-secreting cells resulting in a colored spot were enumerated by microscopic examination and results are expressed semi-quantitatively (−=0-5, +=5-15, ++=15-35, +++=35-50, ++++=>50).

References

Schirmbeck, R., Boehm, W., Melber, K., Reimann, J. (1995). Processing of exogenous heat-aggregated (denatured) and particulate (native) Hepatitis B surface antigen for class I-restricted epitope presentation. J. Imnunol. 155:4676-4684.

1-12. Cancelled

13. A composition for raising an immune response comprising a malaria antigen and an immunostimulatory CpG oligonucleotide.

14. A composition as claimed in claim 13 wherein the antigen is selected from the group of malaria antigens consisting of RTS, RTS*, TRAP and immunologically equivalent derivatives thereof.

15. A composition as claimed in claim 13 wherein the vaccine comprises TRAP or immunologically equivalent derivative and one of RTS or RTS*.

16. A composition as claimed in claim 13 further comprising an aluminum salt, 3 de-O-acylated monophosphoryl lipid A or a saponin adjuvant.

17. A composition as claimed in claim 13 wherein the oligonucleotide comprises two CpG dinucleotides.

18. A composition as claimed in claim 13 wherein the CpG oligonucleotide is between 15-45 nucleotides in length.

19. A composition as claimed in claim 13 wherein the CpG oligonucleotide comprises at least one phosphorothioate internucleotide bond.

20. A composition as claimed in claim 13 wherein the oligonucleotide is selected from the group consisting of oligonucleotides designated as WD1001, WD1002, WD1003, WD1004, WD1005, WD1006, and WD1007.

21. A method for the prevention or amelioration of plasmodium infection in a patient, comprising administering an effective amount of a composition of claim 13 to a patient.

22. A method for the prevention or amelioration of plasmodium infection in a patient, comprising administering an effective amount of a composition of claim 16 to a patient.

23. A method of producing a composition as claimed in claim 13 comprising admixing a malarial antigen and a CpG immunostimulatory oligonucleotide.

24. A method for the prevention or amelioration of plasmodium infection in a patient, comprising administering an effective amount of a CpG oligonucleotide followed after a suitable time by an effective amount of a malaria antigen.
